(19) United States
(12) Patent Application Publication
(10) Pub. No.: US 2026/0355469 A1
(43) Pub. Date: Nov. 12, 2026

(54) APPARATUS AND METHOD FOR USE OF PERFORATED INTRODUCER

(71) Applicant: THE CLEVELAND CLINIC FOUNDATION, Cleveland, OH (US)

(72) Inventor: Torey Hovest, Cleveland, OH (US)

(21) Appl. No.: 19/118,064

(22) PCT Filed: Oct. 5, 2023
(86) PCT No.: PCT/US2023/034510

§ 371 Date: Apr. 3, 2025

Related U.S. Application Data
(60) Provisional application No. 63/413,270, filed on Oct. 5, 2022.

(51) Int. Cl. A61M 25/06; A61M 1/36; A61M 25/00

(52) U.S. Cl. CPC A61M 25/0662; A61M 1/3659; A61M 25/007; A61M 2025/0004

(57) ABSTRACT

A dual lumen drainage cannula (10) configured for use in a VA ECMO system includes a first drainage tube (12) having a proximal end, a distal end, and at least one aperture (18) in at least one wall of the first drainage tube (12) proximate to the distal end of the first drainage tube, and a second drainage tube (14) having a proximal end, a distal end, and at least one aperture (20) in at least one wall of the second drainage tube proximate to the distal end of the second drainage tube. The first drainage tube (12) passes through the second drainage tube (14). The dual lumen drainage cannula (10) also includes a sleeve (16) positioned adjacent to an interior wall of the second drainage tube. The sleeve (16) is formed of a flexible material so as to be expandable and collapsible within the second drainage tube.

### RELATED APPLICATIONS

This application claims priority from U.S. Provisional Application No. 63/413,270, filed 5 Oct. 2022, the subject matter of which is incorporated herein by reference in its entirety for all purposes.

This technology relates to U.S. Patent Application Publication No. 2023/0019859, titled “Apparatus and Method for Use of Fluid Flow Device”, published 19 Jan. 2023 by Kenneth McCurry et al. (hereafter referenced as “the '859 publication”), which is incorporated herein by reference in its entirety for all purposes.

### TECHNICAL FIELD

This disclosure relates to an apparatus and method for use of a perforated introducer and, more particularly, to a method and perforated introducer apparatus for use with extracorporeal membrane oxygenation.

### BACKGROUND

Extracorporeal membrane oxygenation (“ECMO”), also known as extracorporeal life support, is an extracorporeal technique of providing prolonged cardiac and/or respiratory support to persons whose heart and/or lungs are unable to provide an adequate amount of gas exchange and/or perfusion to sustain life. For example, ECMO could be provided to a patient during or after a major surgical procedure, to help reduce the burden on the patient's own body to oxygenate the blood due either to heart or lung dysfunction, or both.

In ECMO, deoxygenated blood is removed from the vasculature of a patient's body via one lumen of a device, is perfused with oxygen and/or otherwise treated in a therapeutic manner, and then is returned to the patient's vasculature (either through a different lumen of the same device or through a different device). An example ECMO system and associated devices are taught by the '859 publication, among others.

FIGS. 1-2 depict a prior art ECMO cannula assembly 100 (which could be a cannula assembly as taught by the '859 publication) as configured for use in a patient's heart. In FIG. 1, the ECMO cannula assembly 100 is configured to facilitate fluid exchange through a patient's right atrium (“RA”). In FIG. 2, the ECMO cannula assembly is configured to facilitate fluid exchange through a patient's pulmonary artery (“PA”). In both Figures, the cannula apparatus 100 may be utilized to remove fluid from, and concurrently or consecutively return fluid to, a vasculature of a patient. The outer sheath 102 is inserted to a target removal site in the patient's body. An infusion cannula 104 could be at least partially held within the outer sheath 102 for concurrent insertion, or the infusion cannula 104 could be absent from the outer sheath 102 until the outer sheath 102 is placed as desired. Once the outer sheath 102 is at the target removal site, the infusion cannula 104 could be inserted (or further inserted) distally into the outer sheath 102—optionally with the aid of an introducer—until a distal infusion cannula end extends out a side access aperture of the outer sheath 102, into the configuration shown in FIGS. 1-2, with a fluid return aperture 106 at a target return site within the patient's vasculature. At that time, the outer sheath 102 can be used to remove fluid from the target removal site (e.g., via fluid removal aperture 108), and the infusion cannula 104 could be used, sequentially and/or simultaneously, to return fluid to the target return site as desired.

Both the outer sheath 102 and the infusion cannula 104 are in fluid communication with an ECMO circuit at least partially outside the patient's body (shown schematically at 110 as an ECMO processing machine) for desired processing, such as oxygenation, of the blood removed from the body through the outer sheath 102 before it is returned to the infusion cannula 104.

FIGS. 3-4 schematically depict a prior art introducer 112 which can be used to position an infusion cannula 104 in a patient's vasculature, optionally with the help of guidewire 114. As is shown in FIG. 3 via the x-ended lines, bloodflow (“BF”) provided to the infusion cannula 104 from the ECMO processing machine 110 is blocked by the introducer 112 from passing through the infusion cannula 104 when the introducer 112 is located therein. As a result, repositioning of the prior art ECMO cannula assembly 100 from the RA-outlet position of FIG. 1 to the PA-outlet position of FIG. 2, with the aid of an introducer 112, cannot be done while still allowing infusion of blood through the infusion cannula 104 to the patient's vasculature via the ECMO circuit. Instead, prior art cannula-moving technique requires that the supply of ECMO blood to the patient be interrupted while the infusion cannula 104 is moved, which can be detrimental to the well-being of the patient in some situations.

### SUMMARY

In an aspect, an introducer is described. An introducer body has longitudinally spaced proximal and distal introducer ends. The introducer body has laterally spaced inner and outer introducer walls. The inner introducer wall defines an introducer lumen. At least one aperture extends laterally through the introducer body between the inner and outer introducer walls to place the introducer lumen in fluid communication with an ambient space therethrough.

### BRIEF DESCRIPTION OF THE DRAWINGS

For a better understanding, reference may be made to the accompanying drawings, in which:

FIG. 1 is a schematic side view of a prior art ECMO device in a first use condition;

FIG. 2 is a schematic side view of a prior art ECMO device in a second use condition;

FIG. 3 is a schematic side view of a prior art introducer which can be used with the ECMO devices of FIGS. 1-2;

FIG. 4 is a schematic side view of the prior art introducer of FIG. 3 in an example use environment;

FIG. 5 is a schematic side view of an introducer according to a first aspect of the present invention;

FIG. 5A is a cross-sectional view taken along line A-A of FIG. 5;

FIG. 6 is a schematic side view of the introducer of FIG. 5 in an example use environment;

FIG. 7 is a schematic side view of an introducer according to a second aspect of the present invention;

FIG. 8 is a schematic side view of the introducer of FIG. 7 in an example use environment;

FIG. 9 is a schematic side view of an introducer according to a third aspect of the present invention; and

FIG. 10 is a schematic side view of the introducer of FIG. 9 in an example use environment.

### DESCRIPTION OF ASPECTS OF THE DISCLOSURE

Unless defined otherwise, all technical and scientific terms used herein have the same meaning as is commonly understood by one of skill in the art to which the present disclosure pertains.

As used herein, the term “subject” can be used interchangeably with the term “patient” and refer to any warm-blooded organism including, but not limited to, human beings, pigs, rats, mice, dogs, goats, sheep, horses, monkeys, apes, rabbits, cattle, farm animals, livestock, etc.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

It will be understood that when an element is referred to as being “on,” “attached” to, “connected” to, “coupled” with, “contacting”, “adjacent”, etc., another element, it can be directly on, attached to, connected to, coupled with, contacting, or adjacent the other element or intervening elements may also be present. In contrast, when an element is referred to as being, for example, “directly on,” “directly attached” to, “directly connected” to, “directly coupled” with, “directly contacting”, or “directly adjacent” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature might not have portions that overlap or underlie the adjacent feature.

Spatially relative terms, such as “under,” “below,” “lower,” “over,” “upper”, “proximal”, “distal”, and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

The invention comprises, consists of, or consists essentially of the following features, in any combination.

FIGS. 5-10 each schematically depict an introducer 500 for cooperatively guiding an infusion cannula 502 for movement as desired inside the vasculature of a patient's body. While the vasculature of a patient is used herein as an example use environment, one of ordinary skill in the art will be able to configure any introducer 500 and/or infusion cannula 502 according to the present invention for a desired use environment (such as, but not limited to, flushing cerebrospinal fluid, ECMO (including pulmonary artery infusion), ECLS, right heart assist or RVAD, wound drainage and flushing, renal dialysis, peritoneal space drainage and flushing, delivery or removal of fluid based therapeutic treatments, and/or other medical and/or non-medical uses).

As shown in FIG. 5, the introducer 500 includes an introducer body 504 having longitudinally spaced proximal and distal introducer ends 506 and 508, respectively. The “longitudinal” direction, as referenced herein, is substantially parallel to arrow “L” in FIG. 5, which coincides with a direction of insertion of the introducer 500 into the patient's body. The cross-sectional view of FIG. 5A illustrates a section of the introducer body 504 as having laterally spaced inner and outer introducer walls 510 and 512, respectively. The “lateral” direction, as used herein, is substantially perpendicular to the longitudinal direction and is substantially within the plane of the page of FIG. 5A. The inner introducer wall 512 defines an introducer lumen 514.

At least one aperture 516 extends laterally through the introducer body 504 between the inner and outer introducer walls 510 and 512 to place the introducer lumen 514 in fluid communication with an ambient space (shown as “A” in FIG. 5A) therethrough. That is, the at least one aperture 516 permits fluid to flow laterally between the introducer lumen 514 and the ambient space A. The aperture(s) 516 may be of any suitable configuration and location upon the introducer body 504 as desired for a particular use application of the introducer 100. For example, one of ordinary skill in the art can readily provide one or more apertures 516 having any desired size, shape, wall configuration (e.g., tapered outward from the introducer lumen 514), location upon the introducer body 504, extent of the introducer body 504 covered, alignment with other aperture(s) 516, and/or any other physical properties, and any of the apertures 516 could have at least one different physical property from any one or more of the other apertures 516.

An introducer coupler, shown schematically at 518, may be located at the proximal introducer end 506 and may be used to attach the introducer 500 to any other desired component and/or may be a standalone handle for assisting a user to manipulate the introducer 500. When present, the introducer coupler 518 may be a hub with at least one of a seal and a hemostatic valve, and may accordingly be configured to prevent backflow of blood out of the introducer coupler 518.

The introducer body 504 may be tapered centrally inward adjacent the distal introducer end 508, as shown in the Figures, to form a nosecone. When present, this tapered “nosecone” shape may have a predetermined size and shape for providing a smooth transition to a distal end of the infusion cannula 502 while tapering forward to more closely match the diameter of a guidewire or small catheter inserted through the introducer 500 for easier tracking and guidance of the introducer 500 within the patient's body.

FIG. 6 shows the introducer 500 ready for placement into the patient's body (or movement/adjustment of position if already located in the patient's body), with the introducer 500 being at least partially inserted into the infusion cannula 502. The introducer 500 and infusion cannula 502 may comprise at least a portion of a system of maintaining blood flow in an ECMO circuit while repositioning at least a portion of the system (that is, without having to block fluid flow through the infusion cannula 502 in order to utilize an introducer 500). The infusion cannula 502 may include proximal and distal cannula ends 520 and 522, respectively, longitudinally spaced by a cannula body 524 defining a cannula lumen 526. The introducer 500 is configured for selective insertion at least partially through the cannula lumen 536, as shown in FIG. 6, with fluid communication between the proximal cannula end 520 and distal introducer end 508 via/through the cannula lumen 536 being at least partially maintained via the at least one aperture 516 and the introducer lumen 514. In other words, and as shown by bloodflow arrows BF in the Figures, blood from the ECMO processing machine 110 (that was previously removed from the patient's body and treated) can be directed into the proximal cannula end 530, where it enters the introducer lumen 514 through one or more apertures 516 adjacent the proximal introducer end 506. The blood can then travel through the introducer lumen 514 and out one or more apertures 516 adjacent the distal introducer end 508, thus maintaining bloodflow from the ECMO circuit to the patient's vasculature while the introducer 500 is assisting with movement of the infusion cannula 502. It is contemplated that the introducer 500 may be configured to fit snugly enough inside the cannula lumen 526 that any apertures 516 located within the cannular lumen 526 are effectively blocked, thus helping direct bloodflow longitudinally out beyond the distal cannula end 522 before a majority of the blood exits the introducer lumen 514 through the apertures 516.

The introducer lumen 514 may be configured to selectively accept at least a portion of a guidewire 528 entirely longitudinally therethrough. For example, and as shown in at least FIG. 6, the system may include the guidewire 528 for selective insertion at least partially through the introducer lumen 514 from the proximal introducer end 506 to a distal aperture 530 located at the distal introducer end 508. This distal aperture 530 may extend substantially coaxially with the introducer lumen 514, and selectively accept at least a portion of the guidewire 528 longitudinally therethrough, so as to form a “straight shot” for the user to employ the guidewire 528 in a known manner to assist with movement of the infusion cannula 502, when the introducer 500 is located at least partially therein.

Optionally, a guidewire lumen 532 may be defined within the introducer lumen 514 by a guidewire tube (shown as optional in dashed line at 534) extending at least partially longitudinally therethrough—for example, if it is desirable to separate the guidewire 528 from the bloodflow.

The infusion cannula 502 may include a bifurcated coupler (shown generally at 536; the coupler could be integrated with, or separately provided to, the infusion cannula 502) at a proximal cannula end 520. The bifurcated coupler 536, when present, may include a first coupler branch 538 configured for selective fluid connection to the ECMO processing machine 110 supplying blood to the Infusion cannula 502. The bifurcated coupler 536 may include a second coupler branch 540 configured to selectively admit the introducer body 504 thereinto (e.g., by being brought at an angle in from a side of the infusion cannula 502). The first and second coupler branches 538 and 540 merge into the cannula lumen 526 at a distalmost end of the bifurcated coupler 536. As is shown in the Figures, the introducer 500 is sufficiently flexible to enter the second coupler branch 540 and then bend along the cannula lumen 526 in order to bolster and guide the structure of the infusion cannula 502 for motion inside the body.

Particularly when the introducer 500 and infusion cannula 502 are being used for an ECMO procedure, or any other filtering or fluid treatment procedure, the removed fluid could be therapeutically treated (e.g., could be oxygenated and/or filtered) after its removal from the vasculature and before its return to the vasculature via the infusion cannula 502. Any desired treatment devices, with appropriate connectors, could be provided to, and/or used with, the introducer 500 and infusion cannula 502 as desired.

As shown in FIG. 5, at least one of the apertures 516 may comprise a void in a repeating or regular lattice pattern formed by the introducer body 504. Though the Figures are not shown to scale here, nor are apertures 516 restricted to predetermined locations along the introducer body 504, the FIG. 5 aperture arrangement is an example of a repeating void/lattice pattern arrangement.

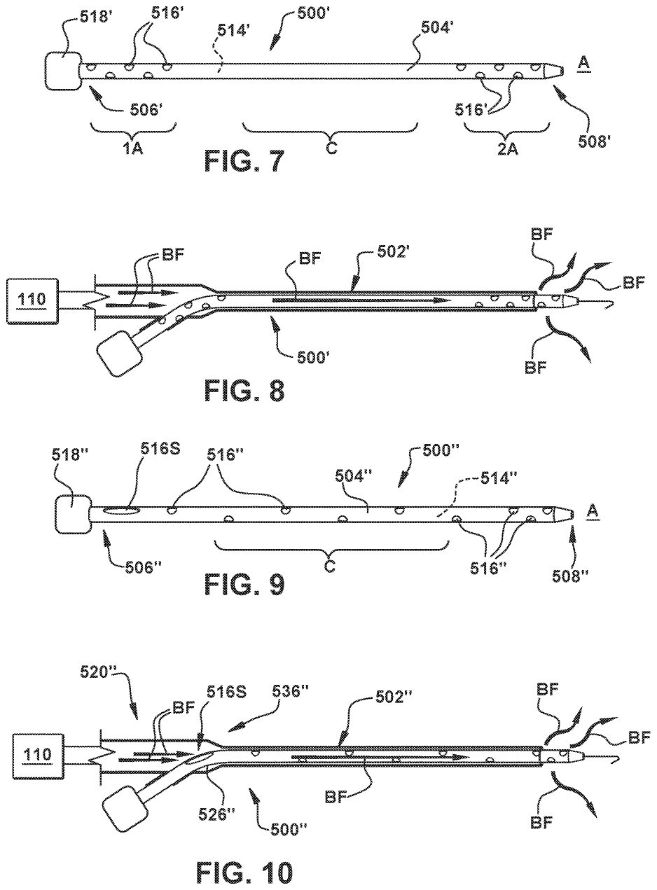

More generally, the “lattice pattern” description may be one specific and nonlimiting example of a configuration of the introducer 500 wherein a plurality of apertures 516 are each spaced apart from one another but are collectively located along a supermajority of a length (in the longitudinal direction) of the introducer body 504. Other arrangements of one or more apertures 516 are available for the introducer 500 configuration, and may readily be provided by one of ordinary skill in the art for a particular use environment. Two other specific and nonlimiting examples of introducer 500 configurations will be discussed in more detail below, with reference to FIGS. 7-10. It is again noted, however, that the Figures of the present application are merely schematic in nature and are not drawn to scale.

In summary, and as shown in the Figures, a plurality of apertures 516 may be longitudinally spaced apart from one another along the introducer body 504. Direct or indirect fluid communication from a fluid source (e.g., ECMO processing machine 110, as shown in the Figures) into the introducer lumen 514 may be provided by a first subset of the plurality of apertures 516 which are located in a first area of the introducer 500. As shown in the Figures, the first subset of the plurality of apertures 516 are located in an area adjacent the proximal introducer end 506, but which are not substantially blocked from inflow by close lateral proximity of the infusion cannula 502 inner wall. Once the stream of fluid (shown here as bloodflow BF) has entered the introducer lumen 514 via the first subset of apertures 516, then fluid communication from the introducer lumen 514 out to the ambient space A (here, at least initially into an area longitudinally distal the distal infusion cannula end 522) may be provided by a second subset of the plurality of apertures 516, wherein the second subset of apertures 516 are located in a second area of the introducer 500, longitudinally spaced from the first area. As shown in the Figures, the second subset of the plurality of apertures 516 are located in an area adjacent the distal introducer end 508, but which are not substantially blocked from outflow by close lateral proximity of the infusion cannula 502 inner wall—for example, the second subset of apertures may be located on a portion of the introducer body 504 which protrudes distally from the distal infusion cannula end 522.

FIGS. 7-8 illustrate a second embodiment of an introducer 500′. The introducer 500′ of FIGS. 7-8 is similar to the introducer 500 of FIGS. 5-6 and therefore, structures of FIGS. 7-8 that are the same as or similar to those described with reference to FIGS. 5-6 have the same reference numbers with the addition of a “prime” mark. Description of common elements and operation similar to those in the previously described first embodiment will not be repeated with respect to the second embodiment, but should instead be considered to be incorporated below by reference as appropriate. For clarity, many reference numbers have been omitted from FIGS. 7-8 that are present for analogous structures in FIGS. 5-6.

In the introducer 500′ shown in FIGS. 7-8, a plurality of first apertures 516′ (labeled generally as “1A”) is located adjacent the proximal introducer end 506′ and a plurality of second apertures 516′ (labeled generally as “2A”) is located adjacent the distal introducer end 508′ A central portion (labeled generally as “C”) of the introducer body 504′, located longitudinally between the plurality of first apertures 1A and the plurality of second apertures 2A, includes no apertures—i.e., is devoid of apertures 516′. For some use environments, the central portion C may comprise at least half of a total longitudinal length of the introducer body 504′. (Though again, it is noted that the drawings herein are not to scale.) Again for some use environments, one of the first and second aperture 1A and 2A pluralities could be used to admit blood (e.g., from the ECMO processing machine 110) to the introducer lumen 514′, and the blood could then travel (such as along bloodflow arrows BF) to egress the introducer lumen 514 through the other of the first and second aperture 1A and 2A pluralities (e.g., to the ambient space A).

In the embodiment of the introducer 500′ shown in FIGS. 7-8, the apertures 516′ are situated toward the distal and proximal introducer ends 508′ and 506′ to help provide rigidity to the introducer 500′ structure, while still maintaining bloodflow into and out of the introducer lumen 514′ during repositioning of the infusion cannula 502′ The apertures 516′ could have substantially the same sizes and densities of placement adjacent the respective proximal and distal introducer ends 506′ and 508′, or could be configured (e.g., larger holes in one area and smaller holes in the other) to assist with achieving desired flow dynamics.

FIGS. 9-10 illustrate a third embodiment of an introducer 500″. The introducer 500″ of FIGS. 9-10 is similar to the introducers 500, 500′ of FIGS. 5-8 and therefore, structures of FIGS. 9-10 that are the same as or similar to those described with reference to FIGS. 5-8 have the same reference numbers with the addition of a double “prime” mark. Description of common elements and operation similar to those in the previously described first and second embodiments will not be repeated with respect to the third embodiment, but should instead be considered to be incorporated below by reference as appropriate. For clarity, many reference numbers have been omitted from FIGS. 9-10 that are present for analogous structures in FIGS. 5-8.

In the introducer 500″ shown in FIGS. 7-8, a plurality of apertures 516″ is located at a plurality of locations along the introducer body 504″. (That is to say, there are multiple apertures 516″, with individual apertures 516″ located at one of multiple locations along the introducer body 504″. An average positional density of the plurality of apertures 516″ is higher adjacent proximal and/or distal introducer ends 506″, 508″ than is an average positional density of the plurality of apertures 516″ at a central portion (shown generally at “C”) of the introducer body 504″. The central portion C comprises at least half of a total longitudinal length of the introducer body 504″. In other words, the apertures 516″ may be “clustered” more closely together adjacent one or both of the proximal and distal introducer ends 506″ and 508″ than the apertures 516″ are “clustered” in the central portion C of the introducer body 504″. In situations with such “clustering” placement of the apertures 516″, one of ordinary skill in the art can readily configure an introducer 500″ as desired for a particular use environment.

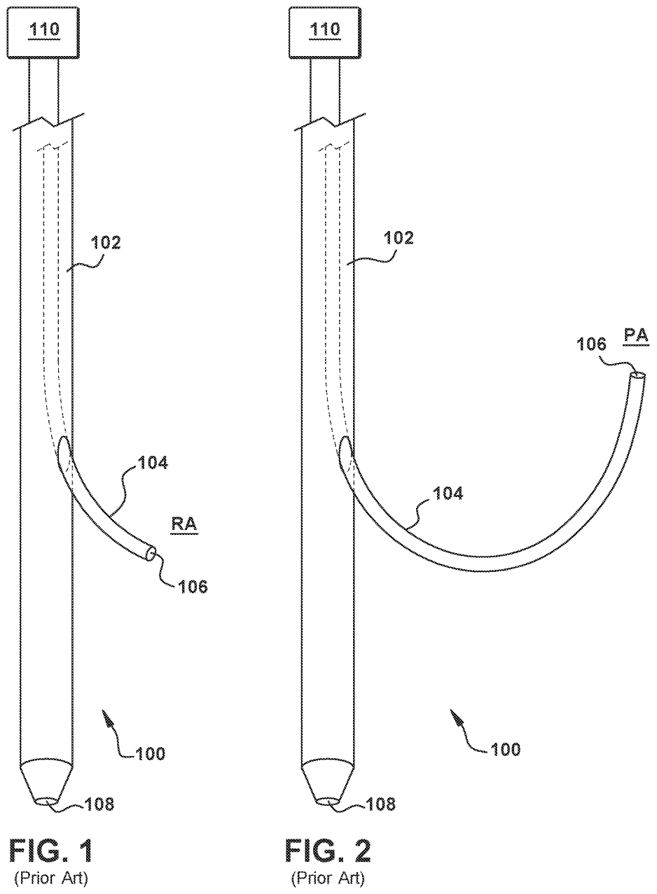

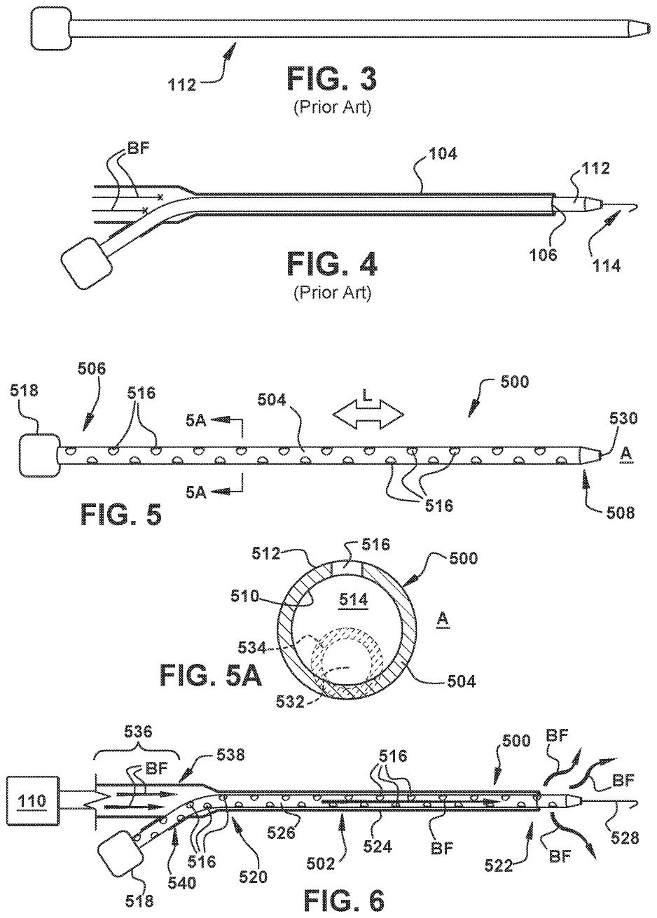

As with any embodiment of the introducer 500, 500′, 500″, the introducer 500″ shown in FIGS. 9-10 may have at least one selected aperture 516S which has a size and/or a perimeter shape which is different from that of at least one other aperture 516″. The selected aperture 516S may have, for example, a larger cross-sectional area than at least one of the other apertures 516″. In FIGS. 9-1, the selected aperture 516S is configured to serve as a cannula aperture 516S adjacent a proximal introducer end 506″. The cannula aperture 516S is located adjacent the proximal cannula end 520″ when the introducer 500″ is inserted at least partially through the cannula lumen 526″, as shown in FIG. 10. When present, the cannula aperture 516S is configured to provide fluid communication between the introducer lumen 514″ and the cannula lumen 526″ in a direction that extends substantially coaxially with the cannula lumen 526″. That is, the cannula aperture 514″ can be substantially aligned with the cannula lumen 526″, as shown in FIG. 10, particularly in situations where the introducer 500″ bends to enter the cannula lumen 526 through a bifurcated coupler 536″ (either integral with, or separately provided to, the infusion cannula 502″).

In order to maintain blood flow in an ECMO processing machine 110 while repositioning at least a portion of the ECMO circuit (here, repositioning the infusion cannula 502 using any embodiment of an introducer 500, 500′, 500″), a method alluded to at various points above may be carried out. A guidewire 528 may be selectively inserted at least partially through the introducer lumen 514 from the proximal introducer end 506 to a distal aperture 530 located at the distal introducer end 508. The introducer 500 may be at least partially inserted through the cannula lumen 526, optionally using a bifurcated coupler 536 or other structure to assist with “side loading” the introducer 500 into the infusion cannula 502. Via allowing bloodflow through at least one aperture 516 and the introducer lumen 514, fluid communication between the proximal cannula end 520 and distal introducer end 508 can be at least partially maintained through the cannula lumen 526.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

I claim:

1. An introducer, comprising:
an introducer body having longitudinally spaced proximal and distal introducer ends, the introducer body having laterally spaced inner and outer introducer walls, the inner introducer wall defining an introducer lumen;
and
at least one aperture extending laterally through the introducer body between the inner and outer introducer walls to place the introducer lumen in fluid communication with an ambient space therethrough.

2. The introducer of claim 1, including an introducer coupler located at the proximal introducer end.

3. The introducer of claim 2, wherein the introducer coupler is a hub with at least one of a seal and a hemostatic valve, the introducer coupler being configured to prevent backflow of blood out of the introducer coupler.

4. The introducer of claim 1, wherein the at least one aperture includes a plurality of apertures spaced apart from one another, the plurality of apertures being collectively located along a supermajority of a length of the introducer body.

5. The introducer of claim 1, wherein the at least one aperture includes a plurality of first apertures located adjacent the proximal introducer end and a plurality of second apertures located adjacent the distal introducer end, wherein a central portion of the introducer body, longitudinally between the plurality of first apertures and the plurality of second apertures, includes no apertures, the central portion comprising at least half of a total longitudinal length of the introducer body.

6. The introducer of claim 1, wherein the at least one aperture includes a plurality of apertures located at a plurality of locations along the introducer body, an average positional density of the plurality of apertures being higher adjacent at least one of the proximal and distal introducer ends than at a central portion of the introducer body, the central portion comprising at least half of a total longitudinal length of the introducer body.

7. The introducer of claim 1, wherein the at least one aperture includes a plurality of apertures, at least one selected aperture having at least one of a size and a perimeter shape which is different from that of at least one other aperture.

8. The introducer of claim 1, wherein the introducer body is tapered centrally inward adjacent the distal introducer end to form a nosecone.

9. The introducer of claim 1, wherein the introducer lumen is configured to selectively accept at least a portion of a guidewire entirely longitudinally therethrough.

10. The introducer of claim 9, wherein a guidewire lumen is defined within the introducer lumen by a guidewire tube extending at least partially longitudinally therethrough.

11. The introducer of claim 1, including a distal aperture located at the distal introducer end and extending coaxially with the introducer lumen.

12. The introducer of claim 11, wherein the distal aperture is configured to selectively accept at least a portion of a guidewire longitudinally therethrough.

13. The introducer of claim 1, wherein at least one of the apertures comprises a void in a repeating lattice pattern formed by the introducer body.

14. A system of maintaining blood flow in an ECMO circuit while repositioning at least a portion of the system, the system comprising:
an Infusion cannula, having proximal and distal cannula ends longitudinally spaced by a cannula body defining a cannula lumen; and
the introducer of claim 1;
wherein the introducer is configured for selective insertion at least partially through the cannula lumen, with fluid communication between the proximal cannula end and distal introducer end through the cannula lumen being at least partially maintained via the at least one aperture and the introducer lumen.

15. The system of claim 14, including a guidewire for selective insertion at least partially through the introducer lumen from the proximal introducer end to a distal aperture located at the distal introducer end.

16. The system of claim 14, wherein the at least one aperture is a plurality of apertures longitudinally spaced apart from one another along the introducer body, wherein fluid communication from an ambient space into the introducer lumen is provided by a first subset of the plurality of apertures located in a first area of the introducer, and wherein fluid communication from the introducer lumen out to the ambient space is provided by a second subset of the plurality of apertures located in a second area of the introducer, longitudinally spaced from the first area.

17. The system of claim 14, wherein the introducer includes a cannula aperture adjacent a proximal introducer end, the cannula aperture being located adjacent the proximal cannula end when the introducer is inserted at least partially through the cannula lumen, the cannula aperture being configured to provide fluid communication between the introducer lumen and the cannula lumen substantially coaxially with the cannula lumen.

18. The system of claim 14, wherein the Infusion cannula includes a bifurcated coupler at a proximal end thereof, the bifurcated coupler including a first coupler branch configured for selective fluid connection to an ECMO processing machine supplying blood to the Infusion cannula, and the bifurcated coupler including a second coupler branch configured to selectively admit the introducer body thereinto, the first and second coupler branches merging into the cannula lumen at a distalmost end of the bifurcated coupler.

19. A method for maintaining blood flow in an ECMO circuit while repositioning at least a portion of the ECMO circuit, the method comprising:
providing an Infusion cannula, having proximal and distal cannula ends longitudinally spaced by a cannula body defining a cannula lumen;
providing the introducer of claim 1;
selectively inserting the introducer at least partially through the cannula lumen; and
at least partially maintaining fluid communication between the proximal cannula end and distal introducer end through the cannula lumen via the at least one aperture and the introducer lumen.

20. The method of claim 19, including selectively inserting a guidewire at least partially through the introducer lumen from the proximal introducer end to a distal aperture located at the distal introducer end.
